(19) United States
(12) Patent Application Publication
(10) Pub. No.: US 2021/0077771 A1
(43) Pub. Date: Mar. 18, 2021

(54) METHOD AND SYSTEM FOR STIMULATING THE NEUROPHYSIOLOGY OF SLEEP

(71) Applicant: Brain Electrophysiology Laboratory Company, LLC, Eugene, OR (US)

(72) Inventors: Don M. Tucker, Eugene, OR (US); Phan Luu, Eugene, OR (US)

(21) Appl. No.: 16/573,182

(22) Filed: Sep. 17, 2019

(51) Int. Cl. A61M 21/02; A61N 1/04; A61N 1/20

(52) U.S. Cl. CPC A61M 21/02; A61N 1/0484; A61N 1/0531; A61N 1/0492; A61N 1/20

(57) ABSTRACT

A method and system for stimulating the neurophysiology of sleep. The method and system are particularly adapted for stimulating the orbitofrontal cortex and at least one of the person's temporal lobes using transcranial electrical stimulation.

### FIELD OF INVENTION

The present invention relates to transcranial electrical stimulation (“TES”) of the brain, and more particularly to a method and system that uses TES for stimulating the neurophysiology of sleep, including deep, or “slow wave,” sleep.

### BACKGROUND

To perform TES, electrodes are attached to the surface of the head and used to impress electrical currents through the head and into the brain, to electrically stimulate the brain at a desired location and thereby to stimulate a desired brain function. The currents are difficult to focus, being free to follow various paths inside the brain, so it is important to optimize the placement of the electrodes to obtain good stimulative efficiency.

Optimizing the placement of the electrodes is typically accomplished by first creating a model of the head, i.e., a model that accounts for the head's anatomical features, particularly its scalp, skull, grey matter, white matter, and cerebrospinal fluid, and that accounts for the head's electrical properties, particularly the electrical impedances of the anatomical features. Then, the optimal positions of the electrodes can be determined by trial and error of running the model, for both optimizing the delivery of current to the desired location, and optimizing avoidance of the delivery of current to other locations.

It is an object of the present invention to adapt this standard TES procedure for use as a means to improve the neurophysiology of sleep, particularly slow wave sleep.

### SUMMARY

Disclosed is a method and system for stimulating the neurophysiology of sleep.

The system includes at least one pair of first electrodes, a headband structure, and a TES controller. The headband structure is adapted for first placing the electrodes of each pair of first electrodes on the person's head in positions that allow for primarily stimulating the person's orbitofrontal cortex and at least one of the person's temporal lobes therewith. And the TES controller is adapted for first controlling the at least one pair of first electrodes so that one of the electrodes of each pair of first electrodes functions as an anode and the other of the electrodes of each pair of first electrodes functions as a cathode for passing respective currents through the electrodes of each pair of first electrodes, to provide for primarily stimulating the person's orbitofrontal cortex and at least one of the person's temporal lobes.

Optionally, the system may include at least one pair of second electrodes, wherein the headband structure is adapted for second placing the electrodes of each pair of second electrodes on the person's head in positions so that the first and second pairs of electrodes all together allow for primarily stimulating the person's orbitofrontal cortex and both of the person's temporal lobes therewith, and wherein the TES controller is adapted for second controlling the at least one pair of second electrodes so that one of the electrodes of each pair of second electrodes functions as an anode and the other of the electrodes of each pair of second electrodes functions as a cathode for passing respective currents through the electrodes of each pair of second electrodes, the first and second controlling together to provide for primarily stimulating the person's orbitofrontal cortex and both of the person's temporal lobes.

To optimize stimulation of the appropriate brain regions, the first placing may result in positioning one of the electrodes of each pair of first electrodes on a portion of the person's forehead located on a first of the left and right sides of the person's head, and the other of the electrodes of each pair of first electrodes on a portion of the person's mastoid area or neck located on the second of the left and right sides of the person's head.

Also to optimize stimulation of the same brain regions, the second placing may result in positioning one of the electrodes of each pair of second electrodes on a portion of the person's forehead located on the second of the left and right sides of the person's head, and the other of the electrodes of each pair of second electrodes on a portion of the person's mastoid area or neck located on the first of the left and right sides of the person's head.

Optionally, in all embodiments, the at least one pair of first electrodes comprising at least three pairs of electrodes.

And optionally, in all embodiments, the system may include an adhesive composition applied to the electrodes of either or both the first and second electrodes.

The method includes providing at least one pair of first electrodes, first placing the first electrodes, and first controlling the first electrodes. The step of first placing places the electrodes of each pair of first electrodes on the person's head in positions that allow for primarily stimulating the person's orbitofrontal cortex and at least one of the person's temporal lobes therewith. And the step of first controlling controls the at least one pair of electrodes so that one of the electrodes of each pair functions as an anode and the other of the electrodes of each pair functions as a cathode for passing respective currents through the electrodes of each pair, said step of first controlling providing for primarily stimulating the person's orbitofrontal cortex and at least one of the person's temporal lobes.

Optionally, the method may include providing at least one pair of second electrodes, second placing the electrodes of each pair of second electrodes on the person's head in positions so that the first and second pairs of electrodes all together allow for primarily stimulating the person's orbitofrontal cortex and both of the person's temporal lobes therewith, and second controlling the at least one pair of second electrodes so that one of the electrodes of each pair of second electrodes functions as an anode and the other of the electrodes of each pair of second electrodes functions as a cathode for passing respective currents through the electrodes of each pair of second electrodes, said steps of first and second controlling together providing for primarily stimulating the person's orbitofrontal cortex and both of the person's temporal lobes.

To optimize stimulation of the appropriate brain regions, the step of first placing may include positioning one of the electrodes of each pair of first electrodes on a portion of the person's forehead located on a first of the left and right sides of the person's head, and the other of the electrodes of each pair of first electrodes on a portion of the person's mastoid area or neck located on the second of the left and right sides of the person's head; and the step of second placing may include positioning one of the electrodes of each pair of second electrodes on a portion of the person's forehead located on the second of the left and right sides of the person's head, and the other of the electrodes of each pair of second electrodes on a portion of the person's mastoid area or neck located on the first of the left and right sides of the person's head.

Also to optimize stimulation of the same brain regions, the step of second placing may include positioning one of the electrodes of each pair of first electrodes on a portion of the person's forehead located on a first of the left and right sides of the person's head, and the other of the electrodes of each pair of first electrodes on a portion of the person's mastoid area or neck located on the second of the left and right sides of the person's head.

Optionally, in all embodiments, the step of first placing may include adhering the electrodes of each pair of first electrodes to the person's skin.

And optionally, in all embodiments, the step of first placing may include fitting a headband structure onto the person's head, to which the electrodes of each pair of electrodes is attached.

It is to be understood that this summary is provided as a means of generally determining what follows in the drawings and detailed description and is not intended to limit the scope of the invention. Objects, features and advantages of the invention will be readily understood upon consideration of the following detailed description taken in conjunction with the accompanying drawings.

### BRIEF DESCRIPTION OF DRAWINGS

FIG. 1 is a plan view of a human brain and six pairs of electrodes positioned relative to the brain for stimulating the neurophysiology of sleep according to the present invention, particularly referencing and indicating three pairs of electrodes that are active.

FIG. 2 is the same as FIG. 1, referencing and indicating the remaining three pairs of electrodes as being active.

FIG. 3 is a left side elevation of a person's head, showing the left side of a headband structure according to the invention, for placing the electrodes shown in FIGS. 1 and 2.

FIG. 4 is a right side elevation of the same person's head as shown in FIG. 3, showing the right side of the headband structure.

FIG. 5 is a schematic plan view of a human brain, equivalent to the views in FIGS. 1 and 2, schematically showing a first example of electrode pairings for left front and right rear pairs of electrodes.

FIG. 6 is a view equivalent to that of FIG. 5, showing a second example of electrode pairings.

FIG. 7 is a view equivalent to that of FIGS. 5 and 6, showing a third example of electrode pairings.

FIG. 8 is a view equivalent to that of FIGS. 5-7, showing a fourth example of electrode pairings.

FIG. 9 is a view equivalent to that of FIGS. 5-8, showing a fifth example of electrode pairings.

### DESCRIPTION OF PREFERRED EMBODIMENTS

The present inventors have discovered that sleep, including deep sleep, can be enhanced by electrically stimulating the temporal lobes and the orbitofrontal cortex of the brain. The inventors have also modeled the human head and determined generally optimal electrode placements for stimulating these particular regions of the brain. These electrode placements have the advantage, for enhancing the neurophysiology of sleep, that they are located on parts of the head that are, generally, free of head hair (as distinct from vellus hair), which allows for more comfortably wearing the electrodes during sleep.

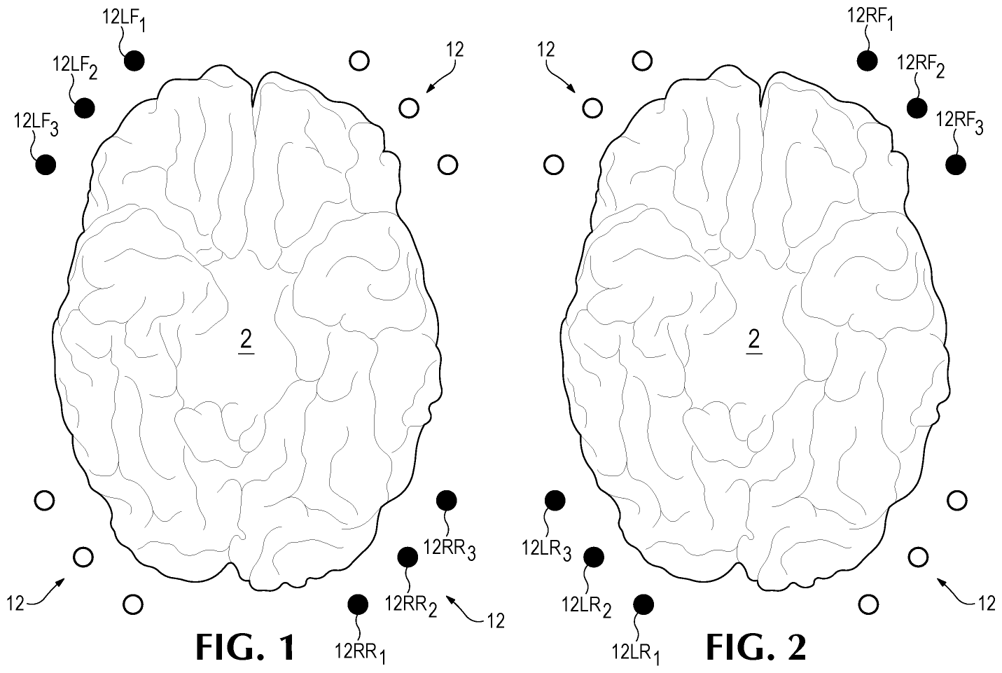

FIGS. 1 and 2 show an example of electrode placements for six pairs of electrodes 12 according to the invention. In FIG. 1, three pairs of the electrodes 12, each having a left-front electrode and a right-rear electrode, are referenced as 12LF₁ and 12RR₁; 12LF₂ and 12RR₂; and 12LF₃ and 12RR₃. These will be referred to as “left-front/right-rear” pairs of electrodes. And in FIG. 2, another three pairs of the electrodes 12, each having a right-front electrode and a left-rear electrode, are referenced as 12RF₁ and 12LR₁; 12RF₂ and 12LR₂; and 12RF₃ and 12LR₃. These will be referred to as “right-front/left-rear” pairs of electrodes.

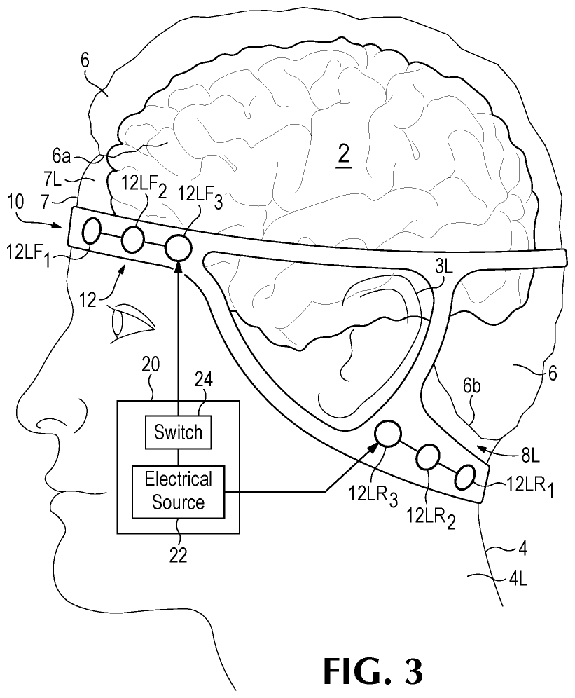

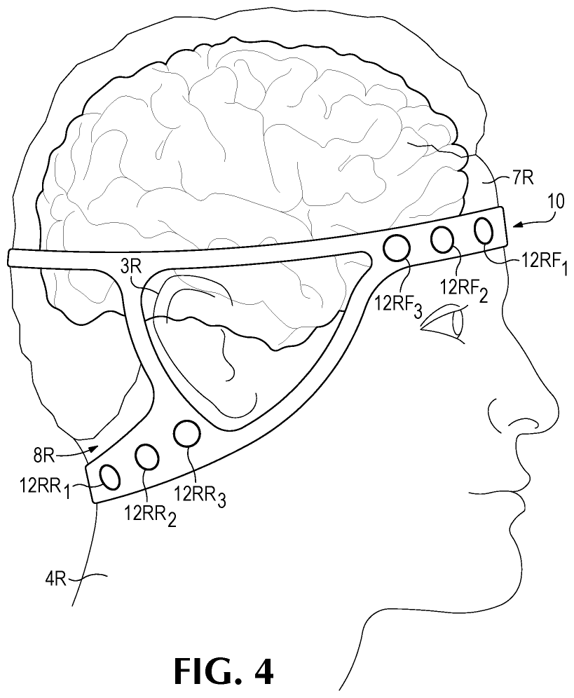

FIGS. 3 and 4 show a headband 10 of the electrodes 12 as it would be worn by a person. FIG. 3 shows the left side of the person's head, and the left side of the headband 10. So FIG. 3 shows the three electrodes 12LF₁, 12LF₂, and 12LF₃ of the left-front/right rear pairs of electrodes, and the three electrodes 12LR₁, 12LR₂, and 12LR₃ of the right-front/left rear pairs of electrodes.

In bilateral symmetry, FIG. 4 shows the right side of the person's head, and the right side of the headband 10. So FIG. 4 shows the three electrodes 12RF₁, 12RF₂, and 12RF₃ of the right-front/left rear pairs of electrodes and the three electrodes 12RR₁, 12RR₂, and 12RR₃ of the left-front/right rear pairs of electrodes.

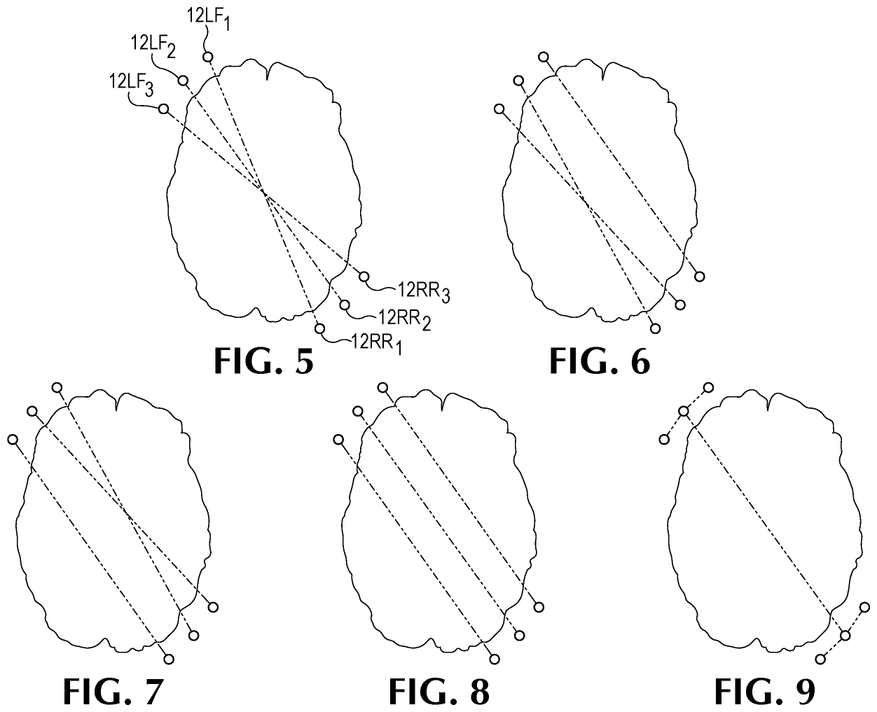

Referring specifically to FIG. 3, the electrodes 12 are controlled by a TES controller 20. TES is well known prior art and need not be discussed in great detail. Basically, for each pair of the electrodes 12, an electrical current is caused to flow from one of the electrodes of the pair, which functions as a “cathode,” to the other of the electrodes of the pair, which functions as an “anode.” The controller 20 includes a multi-channel electrical source 22 to produce the electrical currents by controlling the voltages at the anodes and cathodes of each pair of electrodes, with each pair of electrodes defining a channel. The controller 20 also includes a digital switch 24 that allows for interconnecting the electrodes to form any desired pairings, examples of which are shown in FIGS. 5-9 as discussed further below. An example of such a controller is the “Intan Recording/Stimulation Recorder,” marketed by Intan Technologies LLC of Los Angeles, Calif.

For reference, the person has a brain 2, two ears 3L (FIG. 3) and 3R (FIG. 4), a neck 4, head hair 6, and a forehead 7 defined as being below the person's front head-hair line 6a. The neck 4 has a left side portion 4L (FIG. 3) and a right side portion 4R (FIG. 4); the forehead 7 has a left-side portion 7L (FIG. 3) and a right-side portion 7R (FIG. 4); and there are left-side and right-side areas of the person's head behind the person's ears, above the neck and below the person's rear head-hair line 6b, indicated as 8L (FIG. 3) and 8R (FIG. 4), which will be referred as “mastoid areas” due to their proximity to the mastoid bones.

Generally according to the invention, one of the electrodes of each pair of left-front/right-rear pairs of electrodes is positioned on the left-side forehead portion 7L, and the other is positioned on either or both the right-side mastoid area 8R and the right side portion 4R of the neck 4; and one of the electrodes of each pair of right-front/left-rear pairs of electrodes is positioned on the right-side forehead portion 7R, and the other is positioned on either or both the left-side mastoid area 8L and the left side portion 4L of the neck 4.

For purposes herein, the phrase “mastoid area or neck,” referring to a particular one of the left and right sides of the head, means either or both the mastoid area and the neck on that side of the head.

These electrode placements, provided by the headband 10, are optimized for stimulating the temporal lobes and the orbitofrontal cortex of the brain, and thereby enhancing the neurophysiology of the person's sleep. Because these placements can be on areas of skin that are free of head hair, the electrodes may advantageously be provided with an adhesive as known in the medical arts. For example, adhesive electrodes are commercially available from Leonhard Lang USA, of Inverness Florida, which are marketed as “Skintact F301” solid adhesive gel electrodes.

Use of adhesive electrodes may allow for dispensing with the headband structure 10; however, even if the headband structure is used, adhesive electrodes can provide an advantage of allowing for more accurate electrode placement.

In FIGS. 5-9, the dashed lines identify pairs of electrodes wherein one of the electrodes is a cathode and the other is an anode. FIG. 5 shows the electrode pairings described above. FIGS. 6-9 show alternative electrode pairings. These are just examples of pairings for three pairs of electrodes, it being understood that any number of pairs of electrodes could be used. Generally, the pairing is one electrode positioned at the left front of the head to one electrode positioned at the right rear of the head; and/or one electrode positioned at the right front of the head to one electrode positioned at the left rear of the head.

All the electrode pairs are preferably employed sequentially; e.g., referring to FIG. 5, dining times when the electrode pair 12a₁ and 12b₁ is active, all the remaining pairs are preferably turned off, so that current flow will be limited to flowing between the electrodes of that particular pair. But this is not necessary; for example, the pairings shown in FIG. 9 are indistinguishable from turning all the electrodes shown in the other FIGS. 5-8 on at the same time. In the example of FIG. 9, the three pairs of electrodes are connected together to create, essentially, two large electrodes, which can be advantageous for limiting the current density flowing between the electrodes and therefore decreasing pain caused by the stimulation.

For purposes herein, using any pair of electrodes to pass current therebetween will be referred to as “activating” the pair of electrodes.

Other than (preferably, in most cases) being provided at different times, the pattern of stimulation (e.g., pulses of 0.5-1.0 Hz) used for all the electrodes are typically substantially identical, although this is not essential.

As noted previously, the positioning of the electrodes as described above is optimized for stimulating the temporal lobes (left and/or right) and the orbitofrontal cortex. The electrodes are placed so that they will “primarily” stimulate at these brain regions, which means for purposes herein that the electrodes are placed and controlled by the controller 24 so that one or more of these three regions will be stimulated more than any other region(s) of the brain.

It is to be understood that, while a specific method and system for stimulating the neurophysiology of sleep have been shown and described as preferred, other configurations and methods could be utilized, in addition to those already mentioned, without departing from the principles of the invention.

The teens and expressions which have been employed in the foregoing specification are used therein as tel is of description and not of limitation, and there is no intention in the use of such terms and expressions to exclude equivalents of the features shown and described or portions thereof, it being recognized that the scope of the invention is defined and limited only by the claims which follow.

1. A system for stimulating slow wave sleep in a person, comprising:
at least one pair of first electrodes;
a headband structure adapted for first placing the electrodes of each pair of first electrodes on the person's head in positions that allow for primarily stimulating the person's orbitofrontal cortex and at least one of the person's temporal lobes therewith; and
a TES controller having a user interface enabling a user of the TES controller to configure the TES controller with stimulation parameters for controlling the at least one pair of first electrodes so that one of the electrodes of each pair of first electrodes functions as an anode and the other of each pair of first electrodes functions as a cathode for passing respective currents through the electrodes of each pair of first electrodes so as to stimulate selected portions of the brain, wherein the TES controller is configured with stimulation parameters suitable for primarily stimulating the person's orbitofrontal cortex and at least one of the person's temporal lobes.

2. The system of claim 1, further comprising at least one pair of second electrodes, wherein the headband structure is adapted for second placing the electrodes of each pair of second electrodes on the person's head in positions so that the first and second pairs of electrodes all together allow for primarily stimulating the person's orbitofrontal cortex and both of the person's temporal lobes therewith, and wherein the user interface enables a user of the TES controller to configure the TES controller with selected stimulation parameters for controlling the at least one pair of second electrodes so that one of the electrodes of each pair of second electrodes functions as an anode and the other of the electrodes of each pair of second electrodes functions as a cathode for passing respective currents through the electrodes of each pair of second electrodes so as to stimulate selected portions of the brain, wherein the TES controller is configured with stimulation parameters suitable for primarily stimulating the person's orbitofrontal cortex and both of the person's temporal lobes using the at least one pair of first electrodes and the at least one pair of second electrodes.

3. The system of claim 2, wherein the first placing provides for positioning one of the electrodes of each pair of first electrodes on a portion of the person's forehead located on a first of the left and right sides of the person's head, and the other of the electrodes of each pair of first electrodes on a portion of the person's mastoid area or neck located on the second of the left and right sides of the person's head; and wherein the second placing provides for positioning one of the electrodes of each pair of second electrodes on a portion of the person's forehead located on the second of the left and right sides of the person's head, and the other of the electrodes of each pair of second electrodes on a portion of the person's mastoid area or neck located on the first of the left and right sides of the person's head.

4. The system of claim 1, wherein the first placing provides for positioning one of the electrodes of each pair of first electrodes on a portion of the person's forehead located on a first of the left and right sides of the person's head, and the other of the electrodes of each pair of first electrodes on a portion of the person's mastoid area or neck located on the second of the left and right sides of the person's head.

5. The system of claim 1, the at least one pair of first electrodes comprising at least three pairs of electrodes.

6. The system of claim 2, further comprising an adhesive composition applied to the electrodes of the at least one pair of first electrodes and the electrodes of the at least one pair of second electrodes.

7. The system of claim 1, further comprising an adhesive composition applied to the electrodes of the at least one pair of first electrodes.

8. A method for stimulating slow wave sleep in a person, comprising:
providing at least one pair of first electrodes;
first placing the electrodes of each pair of first electrodes on the person's head in positions that allow for primarily stimulating the person's orbitofrontal cortex and at least one of the person's temporal lobes therewith; and
first controlling the at least one pair of electrodes so that one of the electrodes of each pair functions as an anode and the other of the electrodes of each pair functions as a cathode for passing respective currents through the electrodes of each pair, said step of first controlling providing for primarily stimulating the person's orbitofrontal cortex and at least one of the person's temporal lobes.

9. The method of claim 8, further comprising providing at least one pair of second electrodes, second placing the electrodes of each pair of second electrodes on the person's head in positions so that the first and second pairs of electrodes all together allow for primarily stimulating the person's orbitofrontal cortex and both of the person's temporal lobes therewith, and second controlling the at least one pair of second electrodes so that one of the electrodes of each pair of second electrodes functions as an anode and the other of the electrodes of each pair of second electrodes functions as a cathode for passing respective currents through the electrodes of each pair of second electrodes, said steps of first and second controlling together providing for primarily stimulating the person's orbitofrontal cortex and both of the person's temporal lobes.

10. The method of claim 9, wherein said step of first placing comprises positioning one of the electrodes of each pair of first electrodes on a portion of the person's forehead located on a first of the left and right sides of the person's head, and positioning the other of the electrodes of each pair of first electrodes on a portion of the person's mastoid area or neck located on the second of the left and right sides of the person's head; and said step of second placing comprises positioning one of the electrodes of each pair of second electrodes on a portion of the person's forehead located on the second of the left and right sides of the person's head, and the other of the electrodes of each pair of second electrodes on a portion of the person's mastoid area or neck located on the first of the left and right sides of the person's head.

11. The method of claim 8, wherein said step of first placing comprises positioning one of the electrodes of each pair of first electrodes on a portion of the person's forehead located on a first of the left and right sides of the person's head, and positioning the other of the electrodes of each pair of first electrodes on a portion of the person's mastoid area or neck located on the second of the left and right sides of the person's head.

12. The method of claim 8, wherein said step of first placing comprises adhering the electrodes of each pair of first electrodes to the person's skin.

13. The method of claim 12, wherein said step of first placing comprises fitting a headband structure onto the person's head, to which the electrodes of each pair of electrodes is attached.

14. The method of claim 8, wherein said step of first placing comprises fitting a headband structure onto the person's head, to which the electrodes of each pair of first electrodes is attached.

15. The method of claim 10, wherein a selected one of the at least one pair of first electrodes is employed sequentially relative to a selected one of the at least one pair of second electrodes.

16. The method of claim 9, wherein a selected one of the at least one pair of first electrodes is employed sequentially relative to a selected one of the at least one pair of second electrodes.

17. The system of claim 3, wherein the TES controller is configured with stimulation parameters suitable for primarily stimulating the person's orbitofrontal cortex and at least one of the person's temporal lobes in a frequency range corresponding to that of the slow wave sleep.

18. The system of claim 1, wherein the TES controller is configured with stimulation parameters suitable for primarily stimulating the person's orbitofrontal cortex and at least one of the person's temporal lobes in a frequency range corresponding to that of the slow wave sleep.

19. The method of claim 10, wherein said step of first controlling the at least one pair of electrodes includes primarily stimulating the person's orbitofrontal cortex and at least one of the person's temporal lobes in a frequency range corresponding to that of slow wave sleep.

20. The method of claim 8, wherein said step of first controlling the at least one pair of electrodes includes primarily stimulating the person's orbitofrontal cortex and at least one of the person's temporal lobes in a frequency range corresponding to that of slow wave sleep.
